(19) United States
(12) Patent Application Publication
(10) Pub. No.: US 2010/0056492 A1
(43) Pub. Date: Mar. 4, 2010

(54) COMPOUNDS AND USES THEREOF - 152

(75) Inventors: Peter Bernstein, Wilmington, DE (US); James B. Campbell, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/520,167

(22) PCT Filed: Dec. 19, 2007
(86) PCT No.: PCT/US2007/088055

§ 371 Date: Jun. 19, 2009

Related U.S. Application Data
(60) Provisional application No. 60/870,982, filed on Dec. 20, 2006.

(51) Int. Cl. A61K 31/554; C07D 417/04

(52) U.S. Cl. 514/211.13; 540/551

(57) ABSTRACT

This invention relates to novel compounds having the structural Formula (I) below: and their pharmaceutically acceptable salts or tautomers, compositions and methods of use thereof. These novel compounds provide a treatment or prophylaxis of at least one symptom or condition associated with schizophrenia and other psychotic disorders, dementia and other cognitive disorders, anxiety disorders, mood disorders, sleep disorders, disorders usually first diagnosed in infancy, childhood, or adolescence and neurodegenerative disorders.

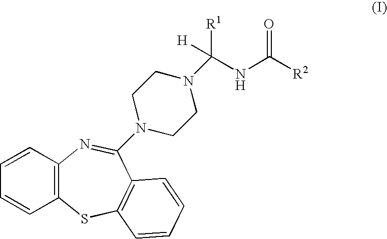

### FIELD OF THE INVENTION

The present invention relates to novel derivatives of 11-piperazin-1-yldibenzo[b,f][1,4]thiazepine, pharmaceutical compositions containing the same, processes for preparing the same, and methods of treating at least one symptom or condition associated with schizophrenia and other psychotic disorders (e.g., psychotic disorder, psychosis), dementia and other cognitive disorders, anxiety disorders (e.g., generalized anxiety disorder), mood disorders (e.g., depressive disorders, major depressive disorders, bipolar disorders including bipolar I and II, bipolar mania, bipolar depression), sleep disorders, disorders usually first diagnosed in infancy, childhood, or adolescence (e.g., attention-deficit disorder and disruptive behavior disorders) and neurodegenerative disorders, comprising administering to a mammal a therapeutically effective amount of a compound of the invention.

### BACKGROUND OF THE INVENTION

One of the goals of antipsychotic drug development has been to develop agents with increased efficacy and safety along with fewer of the side effects commonly associated with older antipsychotic medications. Quetiapine, described in U.S. Pat. No. 4,879,288, has been shown to be effective as a treatment for both the positive (hallucinations, delusions) and negative symptoms (emotional withdrawal, apathy) of psychosis as well as reduction of hostility and aggression. J. Goldstein, Quetiapine Fumarate (Seroquel): a new atypical antipsychotic, 35(3) Drugs of Today 193-210 (1999). Quetiapine is also associated with fewer neurological and endocrine related side effects compared to older agents. In particular, side effects such as EPS, acute dystonia, acute dyskinesia, as well as tardive dyskinesia are less prevalent. Quetiapine has also helped to enhance patient compliance with treatment, ability to function, and overall quality of life while reducing recidivism. P. Weiden et al., Atypical antipsychotic drugs and long-term outcome in schizophrenia, 11 J. Clin. Psychiatry, 53-60, 57 (1996). Because of quetiapine's enhanced tolerability profile, its use is particularly advantageous in the treatment of patients that are hypersensitive to the adverse effects of antipsychotic (such as elderly patients). Quetiapine metabolism has been reported in C. L. Devane et al. Clin. Pharmacokinet., 40(7), 509-522 (2001) proposing an N-dealkylation pathway to the compound 11-piperazin-1-yldibenzo[b,f][1,4]thiazepine (“PDBTZ,” see formula shown below). This compound is also reported by E. Warawa et al. in “Behavioral approach to nondyskinetic dopamine antagonists: identification of Seroquel,” 44 J. Med. Chem., 372-389 (2001) and U.S. Pat. No. 4,879,288. It is now known that 11-piperazin-1-yldibenzo[b,f][1,4]thiazepine is a circulating metabolite of quetiapine in humans.

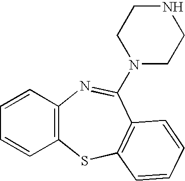

### 11-Piperazin-1-yldibenzo[b,f][1,4]thiazepine (PDBTZ)

Chemical modification of drugs and their metabolites into labile derivatives (prodrugs) with improved physiochemical properties that enable better transport through biological barriers is a useful approach for improving drug delivery. See, e.g., Alexander et. al., J. Med. Chem., 1988, 31, 318-322. The pKa of secondary amines is generally in the range of 10 to 11.2. In the intestine with pH of 7.2, only one-tenth of one percent of these amines is in the uncharged form. It is generally accepted that only the uncharged form of drugs containing these amines can diffuse through the phospholipids bilayer. Therefore, it is evident that these drugs cannot be absorbed in the stomach and are poorly absorbed in the intestine. In addition, these amines are generally good nucleophiles and may also present chemical instability problem in the presence of labile groups in the molecules. Accordingly, there is a need to prepare prodrugs of these amines to circumvent the problems of absorption and instability. It has been reported that the carbamate prodrugs of secondary amines are chemically stable and are readily and quantitative hydroglyzed by esterases to release the parent amines. See, Lin et. al., Biorganic and Medicinal Chemistry Letters, 1997, 7, 2909-2912. Moreover, drugs of secondary amines have been converted in the past to prodrugs such as amides, enamines and Mannich bases for these purposes. See, e.g., Kyncl et al., Adv. Biosci., 1979, 20, 369; Cadwell et al., J. Pharm. Sci., 1971, 60, 1810; Bundgaard et al., J. Pharm. Sci., 1980, 69, 44; and Firestone et al., J. Med. Chem., 1984, 27, 1037.

Because pharmaceutically active compounds and compositions having, for example, improved properties over existing forms are consistently sought, there is an ongoing need for improved forms of existing drug molecules and their active, circulating metabolites. The novel derivatives of 11-piperazin-1-yldibenzo[b,f][1,4]thiazepine described herein are directed toward this and other ends.

### SUMMARY OF THE INVENTION

Provided herein are novel compounds of structural Formula I:

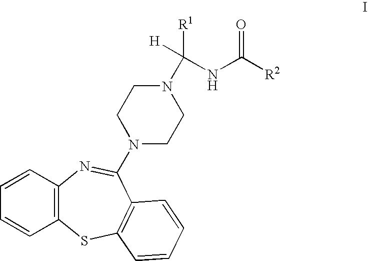

or pharmaceutically acceptable salts thereof, wherein:

R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R³;

R² is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R⁴;

each R³ is, independently, halo, C₁₋₆ alkyl, C₁₋₆ haloalkyl, aryl, cycloalkyl, heteroaryl, heterocycloalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c′}S(═O)₂R^{b}, S(═O)R^{b}, S(═O)NR^{c}R^{d}, S(═O)₂R^{b}, or S(═O)₂NR^{c}R^{d};

each R⁴ is, independently, halo, C₁₋₆ alkyl, C₁₋₆ haloalkyl, aryl, cycloalkyl, heteroaryl, heterocycloalkyl, CN, NO₂, OR^{a′}, SR^{a′}, C(═O)R^{b′}, C(═O)NR^{c′}R^{d′}, C(═O)OR^{a′}, OC(═O)R^{b′}, OC(═O)NR^{c′R}^{d′}, NR^{c′}R^{d′}, NR^{c′}C(═O)R^{b′}, NR^{c′}C(═O)OR^{a′}, NR^{c′}S(═O)₂R^{b′}, S(═O)R^{b′}, S(═O)NR^{c′}R^{d′}, S(═O)₂R^{b′}, or S(═O)₂NR^{c′}R^{d′};

each R^{a} and R^{a′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

each R^{b} and R^{b′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

each R^{c} and R^{d} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{c} and R^{d} together with the N atom to which they are attached form a 4-, 5-, 6-, or 7-membered heterocycloalkyl group; and

each R^{c′} and R^{d′} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{c′} and R^{d′} together with the N atom to which they are attached form a 4-, 5-, 6-, or 7-membered heterocycloalkyl group.

The present invention further provides compositions containing a compound of Formula I described herein, or a pharmaceutically acceptable salt thereof. In some embodiments, the compositions comprise a pharmaceutically acceptable carrier diluent or excipient. In further embodiments, the compositions comprise at least one benzodiazepine, 5-HT_{1A} ligand, 5-HT_{1B} ligand, 5-HT_{1D} ligand, mGluR2A agonist, mGluR5 antagonist, antipsychotic, NK1 receptor antagonist, antidepressant, or serotonin reuptake inhibitor.

The present invention further provides methods of treating at least one symptom or condition associated with schizophrenia and other psychotic disorders (e.g., psychotic disorder, psychosis), dementia and other cognitive disorders, anxiety disorders (e.g., generalized anxiety disorder), mood disorders (e.g., depressive disorders, major depressive disorders, bipolar disorders including bipolar I and II, bipolar mania, bipolar depression), sleep disorders, disorders usually first diagnosed in infancy, childhood, or adolescence (e.g., attention-deficit disorder and disruptive behavior disorders) and neurodegenerative disorders, comprising administering to a mammal a therapeutically effective amount of a compound of the invention.

The present invention further provides a compound of the invention for use in treating the symptoms or conditions provided herein.

The present invention further provides the compounds of the invention for use in the manufacture of a medicament for the treatment of symptoms or conditions provided herein.

The present invention further provides processes for preparing the compounds of formulas described herein.

The present invention further provides methods of delivering 11-piperazin-1-yldibenzo[b,f][1,4]thiazepine by administering to a mammal the compounds of formulas described herein.

### DETAILED DESCRIPTION OF EMBODIMENTS

Provided herein are novel compounds of structural formula I:

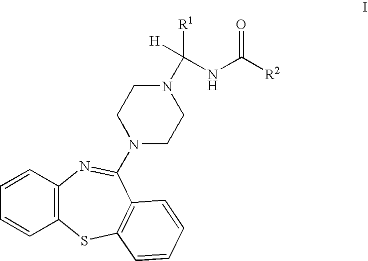

or pharmaceutically acceptable salts thereof, wherein:

R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R³;

R² is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R⁴;

each R³ is, independently, halo, C₁₋₆ alkyl, C₁₋₆ haloalkyl, aryl, cycloalkyl, heteroaryl, heterocycloalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c′}S(═O)₂R^{b}, S(═O)R^{b}, S(═O)NR^{c}R^{d}, S(═O)₂R^{b}, or S(═O)₂NR^{c}R^{d};

each R⁴ is, independently, halo, C₁₋₆ alkyl, C₁₋₆ haloalkyl, aryl, cycloalkyl, heteroaryl, heterocycloalkyl, CN, NO₂, OR^{a′}, SR^{a′}, C(═O)R^{b′}, C(═O)NR^{c′}R^{d′}, C(═O)OR^{a′}, OC(═O)R^{b′}, OC(═O)NR^{c′}R^{d′}, NR^{c′}R^{d′}, NR^{c′}C(═O)R^{b′}, NR^{c′}C(═O)OR^{a′}, NR^{c′}S(═O)₂R^{b′}, S(═O)R^{b′}, S(═O)NR^{c′}R^{d′}, S(═O)₂R^{b′}, or S(═O)₂NR^{c′}R^{d′};

each R^{a} and R^{a′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

each R^{b} and R^{b′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

each R^{c} and R^{d} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{c} and R^{d} together with the N atom to which they are attached form a 4-, 5-, 6-, or 7-membered heterocycloalkyl group; and

each R^{c′} and R^{d′} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{c′} and R^{d′} together with the N atom to which they are attached form a 4-, 5-, 6-, or 7-membered heterocycloalkyl group.

In some embodiments, R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂-(C₁₋₄ alkyl), NHS(═O)₂-(aryl), NHS(═O)₂-(arylalkyl), S(═O)₂-(C₁₋₄ alkyl), S(═O)2-(aryl), S(═O)₂-(arylalkyl), S(═O)₂NH(C₁₋₄ alkyl), S(═O)₂NH(aryl), and S(═O)₂NH(arylalkyl). In some embodiments, R¹ is selected from a subset of its group defined herein. In some embodiments, the substituents on R¹ are selected from a subset of the substitution group defined herein.

In some embodiments, R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, or 3 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, and C₂₋₈ dialkylamino.

In some embodiments, R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl.

In some embodiments, R¹ is H, C₁₋₁₀ alkyl or C₁₋₁₀ haloalkyl. In some further embodiments, R¹ is H, C₁₋₆ alkyl or C₁₋₆ haloalkyl. In yet further embodiments, R¹ is H, C₁₋₄ alkyl, or C₁₋₄ haloalkyl.

In some embodiments, R¹ is H, C₁₋₄ alkyl or C₁₋₄ perhaloalkyl. In some further embodiments, R¹ is H, C₁₋₄ alkyl or C₁₋₄ perfluoroalkyl. In yet further embodiments, R¹ is H, methyl, or trifluoromethyl.

In some embodiments, R² is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂-(C₁₋₄ alkyl), NHS(═O)₂-(aryl), NHS(═O)₂-(arylalkyl), S(═O)₂-(C₁₋₄ alkyl), S(═O)₂-(aryl), S(═O)₂-(arylalkyl), S(═O)₂NH(C₁₋₄ alkyl), S(═O)₂NH(aryl), and S(═O)₂NH(arylalkyl).

In some embodiments, R² is C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, each optionally substituted by 1, 2, 3, 4, or 5 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂-(C₁₋₄ alkyl), NHS(═O)₂-(aryl), NHS(═O)₂-(arylalkyl), S(═O)₂—(C₁₋₄ alkyl), S(═O)₂-(aryl), S(═O)₂-(arylalkyl), S(═O)₂NH(C₁₋₄ alkyl), S(═O)₂NH(aryl), and S(═O)₂NH(arylalkyl).

In some embodiments, R² is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl is optionally substituted by 1, 2, or 3 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, and C₂₋₈ dialkylamino.

In some embodiments, R² is C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, each optionally substituted by 1, 2, or 3 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino, and C₂₋₈ dialkylamino.

In some embodiments, R² is H, C₁₋₁₀ alkyl, or C₁₋₁₀ haloalkyl. In some further embodiments, R² is C₁₋₆ alkyl or C₁₋₆ haloalkyl.

In some embodiments, R² is C₁₋₄ alkyl. In some further embodiments, R² is methyl, ethyl, or isopropyl.

In some embodiments, R¹ is H, C₁₋₄ alkyl or C₁₋₄ haloalkyl; and R² is C₁₋₄ alkyl.

In some embodiments, R¹ is H, C₁₋₄ alkyl or C₁₋₄ perfluoroalkyl; R² is NHC(═O)—R³; and R³ is C₁₋₄ alkyl.

In some embodiments, each R³ is, independently, halo, C₁₋₄ alkyl, C₁₋₄ haloalkyl, aryl, cycloalkyl, heteroaryl, CN, NO₂, OH, C₁₋₄ alkoxy, C₁₋₄ haloalkoxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂—(C₁₋₄ alkyl), NHS(═O)₂-(aryl), NHS(═O)₂-(arylalkyl), S(═O)₂—(C₁₋₄ alkyl), S(═O)₂-(aryl), S(═O)₂-(arylalkyl), S(═O)₂NH(C₁₋₄ alkyl), S(═O)₂NH(aryl), or S(═O)₂NH(arylalkyl).

In some embodiments, each R⁴ is independently halo, C₁₋₄ alkyl, C₁₋₄ haloalkyl, aryl, cycloalkyl, heteroaryl, CN, NO₂, OH, C₁₋₄ alkoxy, C₁₋₄ haloalkoxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂—(C₁₋₄ alkyl), NHS(═O)₂-(aryl), NHS(═O)₂-(arylalkyl), S(═O)₂—(C₁₋₄ alkyl), S(═O)₂-(aryl), S(═O)₂-(arylalkyl), S(═O)₂NH(C₁₋₄ alkyl), S(═O)₂NH(aryl), or S(═O)₂NH(arylalkyl).

In some embodiments, each of R¹, R², R³, and R⁴ is, independently, selected from a subset of any of its groups defined herein respectively. In some embodiments, the substituents on each of R¹, R², R³, and R⁴ are, independently, selected from a subset of any of their corresponding groups defined herein.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆ alkyl, CH₂OH, SO₂H, C₁₋₆ alkyl, OC₁₋₆ alkyl, C(═O)C₁₋₆ alkyl, C(═O)O—C₁₋₆ alkyl, C(═O)NH₂, C(═O)NHC₁₋₆ alkyl, C(═O)N(C₁₋₆ alkyl)2, SO₂C₁₋₆ alkyl, SO₂NH—C₁₋₆ alkyl, SO₂N(C₁₋₆alkyl)₂, NH(C₁₋₆alkyl), N(C₁₋₆ alkyl)₂, NHC(═O)C₁₋₆ alkyl, NC(═O)(C₁₋₆ alkyl)₂, aryl, O-aryl, C(═O)-aryl, C(═O)O-aryl, C(═O)NH-aryl, C(═O)N(aryl)₂, SO₂-aryl, SO₂NH-aryl, SO₂N(aryl)₂, NH(aryl), N(aryl)₂, NC(═O)aryl, NC(═O)(aryl)₂, heterocyclyl, O-heterocyclyl, C(═O)-heterocyclyl, C(═O)O-heterocyclyl, C(═O)NH-heterocyclyl, C(═O)N(heterocyclyl)₂, SO₂-heterocyclyl, SO₂NH-heterocyclyl, SO₂N(heterocyclyl)₂, NH(heterocyclyl), N(heterocyclyl)₂, NC(═O)-heterocyclyl, and NC(═O)(heterocyclyl)₂, or any subset thereof.

A variety of compounds in the present invention may exist in particular stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many stereoisomers of olefins, C═N double bonds, and the like can also be present in the compounds described herein, and all such stable isomers are contemplated in the present invention. Cis and trans isomers of the compounds of the present invention are described and may be isolated as a mixture of isomers or as separated isomeric forms. All chiral, diastereomeric, racemic forms and all stereoisomeric forms of a structure are intended, unless the specific stereochemistry or isomeric form is specifically indicated.

A variety of compounds in the present invention may exist in particular stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. The optically active forms of the compound of the invention may be prepared, for example, by chiral chromatographic separation of a racemate, by synthesis from optically active starting materials or by asymmetric synthesis based on the procedures described thereafter. Many stereoisomers of olefins, C═N double bonds, and the like can also be present in the compounds described herein, and all such stable isomers are contemplated in the present invention. Cis and trans isomers of the compounds of the present invention are described and may be isolated as a mixture of isomers or as separated isomeric forms. All chiral, diastereomeric, racemic forms and all stereoisomeric forms of a structure are intended, unless the specific stereochemistry or isomeric form is specifically indicated.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography. It will also be appreciated that certain compounds of the present invention may exist as geometrical isomers, for example E and Z isomers of alkenes. The present invention includes any geometrical isomer of a compound of the invention.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5, or 6 carbon atoms. Examples of alkyl include, but are not limited to, methyl, ethyl, n-propyl, i-propyl, n-butyl, i-butyl, sec-butyl, t-butyl, pentyl, and hexyl, or any subset thereof. As used herein, “C₁₋₃ alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Examples of alkenyl groups include, but are not limited to, ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Examples of alkynyl groups include, but are not limited to, ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7, or 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3-, and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including, but are not limited to, cyclized alkyl, alkenyl, and alkynyl groups, having the specified number of carbon atoms (wherein the ring comprises 3 to 20 ring-forming carbon atoms). Cycloalkyl groups can include mono- or polycyclic (e.g., having 2, 3, or 4 fused or bridged rings) groups. Examples of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and adamantyl, and the like, or any subset thereof. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Suitable cycloalkyls have from 3 to 10 carbon atoms in their ring structure, and more preferably have 3, 4, 5, or 6 carbons in the ring structure. For example, “C₃₋₆ cycloalkyl” denotes such groups as cyclopropyl, cyclobutyl, cyclopentyl, or cyclohexyl.

“Counterion” is used to represent a small, negatively or positively charged species such as chloride (Cl⁻), bromide (Br⁻), hydroxide (OH⁻), acetate (CH₃COO⁻), sulfate (SO₄²⁻), tosylate (CH₃-phenyl-SO₃⁻), benezensulfonate (phenyl-SO₃⁻), sodium ion (Na⁺), potassium (K⁺), ammonium (NH₄⁺), and the like, or any subset thereof.

As used herein, the term “heterocyclyl” or “heterocyclic” or “heterocycle” refers to a ring-containing monovalent and divalent structures having one or more heteroatoms, independently selected from N, O, and S, as part of the ring structure and comprising from 3 to 20 atoms in the rings, or 3- to 7-membered rings. The number of ring-forming atoms in heterocyclyl are given in ranges herein. For example, C₅₋₁₀ heterocyclyl refers to a ring structure comprising from 5 to 10 ring-forming atoms wherein at least one of the ring-forming atoms is N, O, or S. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, and xanthenyl, or any subset thereof.

As used herein, “heteroaryl” refers to an aromatic heterocycle (wherein the ring comprises up to about 20 ring-forming atoms) having at least one heteroatom ring member such as sulfur, oxygen, or nitrogen. Heteroaryl groups include monocyclic and polycyclic (e.g., having 2, 3, or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, and indolinyl, and the like, or any subset thereof. In some embodiments, the heteroaryl group has from 1 to about 20 carbon atoms, and in further embodiments from about 3 to about 20 carbon atoms. In some embodiments, the heteroaryl group contains from 3 to about 14, from 4 to about 14, from 3 to about 7, or from 5 to 6 ring-forming atoms. In some embodiments, the heteroaryl group has from 1 to about 4, from 1 to about 3, or from 1 to 2 heteroatoms. In some embodiments, the heteroaryl group has 1 heteroatom.

As used herein, “heterocycloalkyl” refers to non-aromatic heterocycles (wherein the ring comprises about 3 to about 20 ring-forming atoms) including cyclized alkyl, alkenyl, and alkynyl groups where one or more of the ring-forming carbon atoms is replaced by a heteroatom such as an O, N, or S atom. Hetercycloalkyl groups can be mono or polycyclic (e.g., both fused and spiro systems). Suitable “heterocycloalkyl” groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, and imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains from 3 to about 14, from 3 to about 7, or from 5 to 6 ring-forming atoms. In some embodiments, the heterocycloalkyl group has from 1 to about 4, from 1 to about 3, or from 1 to 2 heteroatoms. In some embodiments, the heterocycloalkyl group contains from 0 to 3 double bonds. In some embodiments, the heterocycloalkyl group contains from 0 to 2 triple bonds.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy, and propargyloxy, or any subset thereof. Similarly, “alkylthio” or “thioalkoxy” represent an alkyl group as defined above with the indicated number of carbon atoms attached through a sulphur bridge.

As used herein, the term “carbonyl” is art recognized and includes the —C(═O) groups of such moieties as can be represented by the general formula:

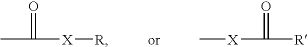

wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to the —S(═O)₂— of a moiety that can be represented by the general formula:

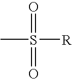

wherein R is represented by, but not limited to, hydrogen, alkyl, cycloalkyl, alkenyl, aryl, heteroaryl, aralkyl, or heteroaralkyl.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “halo” or “halogen” includes fluoro, chloro, bromo, and iodo, or any subset thereof.

As used herein, “haloalkyl” refers to an alkyl group having one or more halogen substituents. Examples of haloalkyl groups include, but are not limited to, CF₃, C₂F₅, CH₂CF₃, CHF₂, CCl₃, CHCl₂, and C₂Cl₅, and the like, or any subset thereof. The term “perhaloalkyl” is intended to denote an alkyl group in which all of the hydrogen atoms are replaced with halogen atoms. One example of perhaloalkyl is CCl₃ or CF₃. The term “perfluoroalkyl” is intended to denote an alkyl group in which all of the hydrogen atoms are replaced with fluorine atoms. One example of perhaloalkyl is CF₃ (i.e., trifluoromethyl).

As used herein, “alkoxy” or “alkyloxy” refers to an —O-alkyl group. Examples of alkoxy groups include, but are not limited to, methoxy, ethoxy, propoxy (e.g., n-propoxy and isopropoxy), t-butoxy, and the like, or any subset thereof.

As used here, “haloalkoxy” refers to an —O-haloalkyl group. An example of a haloalkoxy group is OCF₃.

As used herein, “aryloxy” refers to —O-aryl. An example of a heteroaryloxy is phenoxy.

As used herein, “heteroaryloxy” refers to —O-heteroaryl. An example of a heteroaryloxy is pyridine-2-yloxy [i.e., —O-(pyridine-2-yl)].

As used herein, “arylalkyl” refers to C₁₋₁₀ alkyl substituted by aryl and “cycloalkylalkyl” refers to C₁₋₁₀ alkyl substituted by cycloalkyl. An example of a arylalkyl group is benzyl.

As used herein, “heteroarylalkyl” refers to C₁₋₁₀ alkyl substituted by heteroaryl and “heterocycloalkylalkyl” refers to C₁₋₁₀ alkyl substituted by heterocycloalkyl.

As used herein, “arylalkyloxy” refers to —O-(arylakyl) and “heteroarylalkyloxy” refers to —O-(heteroarylakyl). An example of an arylalkyloxy group is benzyloxy and an example of a heteroarylalkyloxy group is (pyridin-2-yl)-methoxy.

As used herein, some substituents are described in a combination of two or more groups. For example, the expression of “C(═O)—C₃₋₉ cycloalkylR^{d}” is meant to refer to a structure:

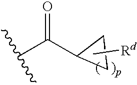

wherein p is 1, 2, 3, 4, 5, 6, or 7 (i.e., C₃₋₉ cycloalkyl); the C₃₋₉ cycloalkyl is substituted by R^{d}; and the point of attachment of the “C(═O)—C₃₋₉ cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include, but are not limited to, esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include, but are not limited to, ²H (also written as D for deuterium), ³H (also written as T for tritium), ¹¹C, ¹³C, ¹⁴C, ¹³N, ¹⁵N, ¹⁵O, ¹⁷, ¹⁸O, ¹⁸F, ³⁵S, ³⁶Cl, ⁸²Br, ⁷⁵Br, ⁷⁶Br, ⁷⁷Br, 123I, 124I, ¹²⁵I, and ¹³¹I, or any subset thereof. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S, and ⁸²Br.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg, and Ca, and with organic amines such as triethylamine and Tris(2-hydroxyethyl)amine. Salts can be formed between compounds with basic groups, e.g. amines, with inorganic acids such as, for example, hydrochloric acid, phosphoric acid, or sulfuric acid, or organic acids such as, for example, acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include, but are not limited to, salts formed with hydrochloric, hydrobromic, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, tartaric, acetic, propanoic, butanoic, malonic, glucuronic, and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine, or any subset thereof. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of Deady (Syn. Comm., 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR, wherein R is an ester substituent, for example, a C₁₋₇ alkyl group, a C₃₋₂₀ heterocyclyl group, or a C₅₂₀ aryl group, preferably a C₁₇ alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include, but are not limited to, those of the formula —C(═O)OR wherein R is: C₁₋₇alkyl (e.g., Me, Et, -nPr, -iPr, -nBu, -sBu, -iBu, tBu); C₁₋₇aminoalkyl (e.g., aminoethyl; 2-(N,N-diethylamino)ethyl; 2(4morpholino)ethyl); and acyloxy-C₁₋₇alkyl (e.g., acyloxymethyl; acyloxyethyl; pivaloyloxymethyl; acetoxymethyl; 1acetoxyethyl; 1-(1-methoxy-1-methyl)ethyl-carbonyloxyethyl; 1-(benzoyloxy)ethyl; isopropoxy-carbonyloxymethyl; 1isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1(4-tetrahydropyranyl)carbonyloxyethyl), or any subset thereof.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of tautomeric forms and references to compounds include all such forms. For the avoidance of doubt, where a compound can exist in one of several tautomeric forms and only one is specifically described or shown, all others are nevertheless embraced by the scope of this invention.

Compounds of the present invention also include pharmaceutically acceptable salts and tautomers of the compounds of any of the formulas described herein. Compounds of the invention further include hydrates and solvates. It will also be understood that certain compounds of the present invention may exist in solvated, for example hydrated, as well as unsolvated forms. It will further be understood that the present invention encompasses all such solvated forms of the compounds of the invention.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, polymorphic forms of the compounds, solvates (e.g. hydrates), or lipids, and compounds having coupling partners and protecting groups (such as protecting groups for amino and/or hydroxyl group).

A compound of Formula I, or a pharmaceutically acceptable salt or solvate thereof, or a pharmaceutical composition or formulation comprising a compound of Formula I or its pharmaceutically acceptable salt or solvate, can be administered concurrently, simultaneously, sequentially or separately with another compound or compounds selected from the following:

(i) antidepressants such as, for example, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robaizotan, sertraline, sibutramine, thionisoxetine, tranylcypromaine, trazodone, trimipramine, and venlafaxine, and equivalents and pharmaceutically active isomer(s) and/or metabolite(s) thereof;

(ii) atypical antipsychotics including, for example, quetiapine and pharmaceutically active isomer(s) and/or metabolite(s) thereof;

(iii) antipsychotics including, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, and ziprasidone, and equivalents and pharmaceutically active isomer(s) and/or metabolite(s) thereof;

(iv) anxiolytics including, for example, alnespirone, azapirones, benzodiazepines, and barbiturates, and equivalents and pharmaceutically active isomer(s) and/or metabolite(s) thereof. Examples of anxiolytics include, but are not limited to, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, and zolazepam;

(v) anticonvulsants including, for example, carbamazepine, valproate, lamotrogine, and gabapentin, and equivalents and pharmaceutically active isomer(s) and/or metabolite(s) thereof;

(vi) Alzheimer's therapies including, for example, donepezil, memantine, and tacrine, and equivalents and pharmaceutically active isomer(s) and/or metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, and Dopamine agonists and inhibitors of neuronal nitric oxide synthase, and equivalents and pharmaceutically active isomer(s) and/or metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, and zomitriptan, and equivalents and pharmaceutically active isomer(s) and/or metabolite(s) thereof;

(ix) stroke therapies including, for example, abciximab, activase, NXY-059, citicoline, crobenetine, desmoteplase, repinotan, and traxoprodil, and equivalents and pharmaceutically active isomer(s) and/or metabolite(s) thereof;

(x) urinary incontinence therapies including, for example, darafenacin, falvoxate, oxybutynin, propiverine, robalzotan, solifenacin, and tolterodine, and equivalents and pharmaceutically active isomer(s) and/or metabolite(s) thereof;

(xi) neuropathic pain therapies including, for example, gabapentin, lidoderm, and pregablin, and equivalents and pharmaceutically active isomer(s) and/or metabolite(s) thereof;

(xii) nociceptive pain therapies such as, for example, celecoxib, etoricoxib, lumiracoxib, rofecoxib, valdecoxib, diclofenac, loxoprofen, naproxen, and paracetamol, and equivalents and pharmaceutically active isomer(s) and/or metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, and zolpidem, and equivalents and pharmaceutically active isomer(s) and/or metabolite(s) thereof;

(xiv) mood stabilizers including, for example, carbamazepine, divalproex, gabapentin, lamotrigine, lithium, olanzapine, quetiapine, valproate, valproic acid, and verapamil, and equivalents and pharmaceutically active isomer(s) and/or metabolite(s) thereof;

(xv) 5HT_{1B} ligands such as, for example, compounds disclosed in WO99/05134 and WO02/08212;

(xvi) mGluR2 agonists;

(xvii) alpha 7 nicotinic agonists such as, for example, compounds disclosed in WO96/006098, WO97/030998, WO99/003859, WO00/042044, WO01/029034, WO01/160821, WO01/136417, WO02/096912, WO03/087102, WO03/087103, WO03/087104, WO04/016617, WO04/016616, and WO04/019947;

(xviii) chemokine receptor CCR1 inhibitors; and

(xix) delta opioid agonists such as, for example, compounds disclosed in WO97/23466 and WO02/094794.

Such combination products employ the compounds of this invention within the dosage range described herein and the other pharmaceutically active agent within approved dosage ranges and/or the dosage such as described in the publication reference.

In some embodiments, the present invention provides a compound of any of the formulas described herein or a pharmaceutically acceptable salt thereof for the therapeutic treatment (including prophylactic treatment) of mammals including humans, and it is normally formulated in accordance with standard pharmaceutical practice as a pharmaceutical composition. The present invention further encompasses pharmaceutical compositions containing one or more compounds of the present invention.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For preparing pharmaceutical compositions from the compounds of this invention, inert, pharmaceutically acceptable carriers can be either solid or liquid. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Solid form compositions include powders, tablets, dispersible granules, capsules, cachets, and suppositories. A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

In further embodiments, the pharmaceutical composition includes the compound of the invention in combination with a pharmaceutically acceptable carrier and at least one further active ingredient. Examples of further active ingredients include, but are not limited to, benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, and serotonin reuptake inhibitors.

The pharmaceutical compositions of the invention can accordingly be obtained by conventional procedures using conventional pharmaceutical excipients. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. Pharmaceutical compositions intended for oral use can further contain, for example, one or more coloring, sweetening, flavoring and/or preservative agents.

The composition of the invention can be administered by any route including orally, intramuscularly, subcutaneously, topically, intranasally, intraperitoneally, intrathoracially, intravenously, epidurally, intrathecally, intracerebroventricularly and by injection into the joints.

The amount of active ingredient that is combined with one or more excipients to produce a single dosage form will necessarily vary depending upon the host treated and the particular route of administration. The size of the dose for therapeutic or prophylactic purposes of the active compound(s) will naturally vary according to the nature and severity of the symptoms or conditions, the age and sex of the animal or patient and the route of administration, according to well known principles of medicine.

The present invention further provides methods of treating at least one symptom or condition associated with schizophrenia and other psychotic disorders (e.g., psychotic disorder, psychosis), dementia and other cognitive disorders, anxiety disorders (e.g., generalized anxiety disorder), mood disorders (e.g., depressive disorders, major depressive disorders, bipolar disorders including bipolar I and II, bipolar mania, bipolar depression), sleep disorders, disorders usually first diagnosed in infancy, childhood, or adolescence (e.g., attention-deficit disorder and disruptive behavior disorders) and neurodegenerative disorders comprising administering to a mammal a pharmaceutically effective amount of the salt of the invention or composition containing one or more of the same. In some embodiments, the symptoms and conditions include, but are not limited to anxiety, agitation, hostility, panic, eating disorder, affective symptom, mood symptom, negative and positive psychotic symptom commonly associated with psychosis, and neurodegenerative disorder.

The present invention further provides methods of treating at least one symptom or condition associated with schizophrenia and other psychotic disorders (e.g., psychotic disorder, psychosis), dementia and other cognitive disorders, anxiety disorders (e.g., generalized anxiety disorder), mood disorders (e.g., depressive disorders, major depressive disorders, bipolar disorders including bipolar I and II, bipolar mania, bipolar depression), sleep disorders, disorders usually first diagnosed in infancy, childhood, or adolescence (e.g., attention-deficit disorder and disruptive behavior disorders) and neurodegenerative disorders comprising administering to a mammal a pharmaceutically effective amount of a compound of the invention, or composition containing one or more of the same, and a therapeutically effective amount of at least one other therapeutically active agent selected from benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, and serotonin reuptake inhibitors.

Exemplary benzodiazepines include, but are not limited to, adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, and triazolam, and equivalents thereof.

Exemplary 5-HT_{1A} and/or 5HT_{1B} ligands include, but are not limited to, buspirone, alnespirone, elzasonan, ipsapirone, gepirone, and zopiclone, and equivalents thereof.

Exemplary mGluR2 agonists include, but are not limited to, (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants include, but are not limited to, maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics include, but are not limited to, clozapine, risperidone, quetiapine, olanzapine, amisulpride, sulpiride, zotepine, chlorpromazine, haloperidol, ziprasidone, and sertindole.

Administration of two or more active agents can be carried out in combination, e.g., as part of the same pharmaceutical composition, or separately (e.g., serially or consecutively) as part of an appropriate dose regimen designed to obtain the benefits of combination therapy. The appropriate dose regimen, the amount of each dose of an active agent administered, and the specific intervals between doses of each active agent will depend upon the subject being treated, the specific active agent being administered and the nature and severity of the specific disorder or condition being treated.

In general, the compounds of this invention, when used as either a single active agent or when used in combination with another active agent, will be administered to a subject in an amount up to about 750 mg per day (e.g., 1 mg to 600 mg per day), in single or divided doses. Such compounds may be administered on a regimen of up to 6 times per day, preferably 1 to 4 times per day. Variations can occur depending upon the subject being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect, provided that such larger doses are first divided into several small doses for administration throughout the day.

In some embodiments, the compound of the invention is administered as a predetermined dosage to a mammal between one and four times a day, wherein the predetermined dosage is between 1 mg and 600 mg.

The present invention also provides a method of treating the symptoms or conditions provided herein comprising the step of administering an initial predetermined dosage of a compound of the invention to a human patient twice a day, wherein the predetermined dosage is between 1 mg and 30 mg with increases in increments of 1 to 50 mg twice daily on the second and third day as tolerated. Thereafter, further dosage adjustments can be made at intervals of 2 days or greater.

The present invention further provides a method of delivering 11-piperazin-1-yldibenzo[b,f][1,4]thiazepine to a mammal. In some embodiments, the present invention provides a method of delivering 11-piperazin-1-yldibenzo[b,f][1,4]thiazepine comprising administering to a mammal a therapeutically effective amount of a compound of the present invention. In some embodiments, the present invention provides a method of delivering 11-piperazin-1-yldibenzo[b,f][1,4]thiazepine comprising administering to a mammal a composition which comprises a therapeutically effective amount of a compound of the present invention.

In some embodiments, the present invention provides a method of delivering 11-piperazin-1-yldibenzo[b,f][1,4]thiazepine in the treatment of at least one symptom or condition associated with schizophrenia and other psychotic disorder, dementia and other cognitive disorder, anxiety disorder, mood disorder, sleep disorder, disorder usually first diagnosed in infancy, childhood, or adolescence, and neurodegenerative disorder, comprising administering to a mammal a therapeutically effective amount of a compound of the present invention, or a composition which comprises a therapeutically effective amount of a compound of the present invention.

A “therapeutically effective amount” refers to amount of active compound or pharmaceutical agent that elicits the biological or medicinal response in a tissue, system, animal, individual or human that is being sought by a researcher, veterinarian, medical doctor or other clinician and can be readily determined by a clinician by using numerous methods already known in the art, an example of which is the BPRS cluster score that can be used to assess levels of hostility and positive symptoms.

The term “treating” within the context of the present invention is meant to encompass the administration of a therapeutically effective amount of a compound of the present invention to mitigate or inhibit either a pre-existing disease state, acute or chronic, or a recurring symptom or condition. Also encompassed are prophylactic therapies for prevention of recurring conditions and continued therapy for chronic disorders.

The term “mammal” is meant to refer to any warm-blooded animal, preferably a human. In some embodiments, the mammal is in need of treatment because it is suffering from or prone to developing one or more of the symptoms, diseases or disorders described above.

In order that the invention disclosed herein may be more efficiently understood, examples are provided below. It should be understood that these examples are for illustrative purposes only and are not to be construed as limiting the invention in any manner.

### Synthesis

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. The starting materials and precursors used in the processes described herein were either commercially available or readily prepared by established organic synthesis methods. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods should then be used.

As shown in Scheme 1, a novel Mannich-base-typed compound of the present invention (formula 7-4) can be synthesized by reacting PDBTZ 1-1 with an aldehyde 1-2 (wherein R¹ can be H, alkyl, cycloalkyl, aryl, hetereoaryl, heterocylcloalky and the like) and a nucleophile such as an amide 1-3 (wherein R² can be alkyl, cycloalkyl, aryl, hetereoaryl, heterocylcloalky and the like) under suitable conditions such as reflux. For example, PDBTZ, formaldehyde aqueous solution and an amide 1-3 can be heated to reflux in an alcohol solvent such as ethanol to form a compound of formula 1-4 wherein R¹ is H.

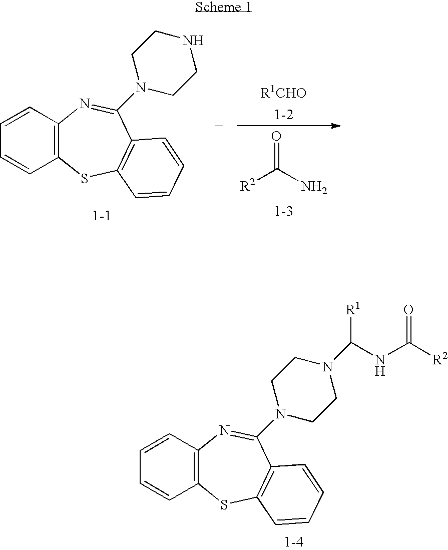

It should noted that in all of the schemes described herein, if there are functional (reactive) groups present on a substituent group such as R¹, R², etc., further modification can be made if appropriate and/or desired. For example, a CN group can be hydrolyzed to afford an amide group; a carboxylic acid can be converted to an amide; a carboxylic acid can be converted to a ester, which in turn can be reduced to an alcohol, which in turn can be further modified. In another example, an OH group can be converted into a better leaving group such as mesylate, which in turn is suitable for nucleophilic substitution, such as by CN. One skilled in the art will recognize further such modifications. Thus, a compound of formula I (such as compound 1-4 of Scheme 1) having a sustituent which contains a function group can be converted to another compound of formula I having a different substituent group.

As used herein, the term “reacting” refers to the bringing together of designated chemical reactants such that a chemical transformation takes place generating a compound different from any initially introduced into the system. Reacting can take place in the presence or absence of solvent.

As used herein, the term “leaving group” refers to a moiety that can be displaced by another moiety, such as by nucleophilic attack, during a chemical reaction. Leaving groups are well known in the art and include, for example, halogen, hydroxy, alkoxy, —O(C═O)R^{a}, —OSO₂—R^{b}, and —OSi(R^{c})₃ wherein R^{a} can be C₁₋₈ alkyl, C₃₋₇ cycloalkyl, aryl, heteroaryl, or heterocycloalkyl, wherein R^{b} can be C₁₋₈ alkyl, aryl (optionally substituted by one or more halo, cyano, nitro, C₁₋₄ alkyl, C₁₋₄ haloalkyl, C₁₋₄ alkoxy, or C₁₋₄ haloalkoxy), or heteroaryl (optionally substituted by one or more halo, cyano, nitro, C₁₋₄ alkyl, C₁₋₄ haloalkyl, C₁-C₄ alkoxy, or C₁₋₄ haloalkoxy), and wherein R^{c} can be C₁₋₈ alkyl. Examples of leaving groups include, but are not limited to, chloro, bromo, iodo, 4-nitrophenylcarbonate, mesylate, tosylate, trimethylsilyl, and the like.

### EXAMPLES

### Example 1

### N-(4-Dibenzo[b,f][1,4]thiazepin-11-yl-piperazin-1-ylmethyl)-acetamide

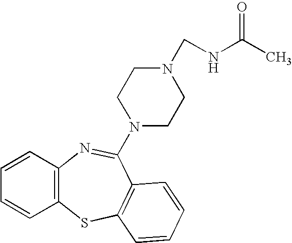

A mixture of 11-iperazin-1-yldibenzo[b,f][1,4]thiazepine (“PDBTZ”, 1 mmol), 37% aqueous formaldehyde (1 mmol), and acetamide (1 mmol) in ethanol (5 mL) is refluxed for four hours. The reaction mixture is evaporated, the residue resuspended in water, and the resulting aqueous material extracted with chloroform. The organic portion is washed (water, brine), dried (sodium sulfate), and evaporated. The crude material is purified by flash chromatography to provide the title compound.

### Example 2

### N-[1-(4-Dibenzo[b,f][1,4]thiazepin-11-yl-piperazin-1-yl)-ethyl]-2,2,2-trifluoro-acetamide

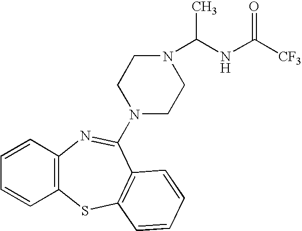

A mixture of PDBTZ (1 mmol), acetaldehyde (1 mmol), and 2,2,2-trifluoro-acetamide (1 mmol) in ethanol (5 mL) is refluxed for four hours. The reaction mixture is evaporated, the residue resuspended in water, and the resulting aqueous material extracted with chloroform. The organic portion is washed (water, brine), dried (sodium sulfate), and evaporated. The crude material is purified by flash chromatography to provide the title compound.

### Example 3

### Murine Assays

An assessment of dopamine antagonism for the compounds and compositions of the invention can be made in rodent models. The methods and procedures used can be found in J. Med. Chem., 2001, 44, 372-389, which is incorporated herein by reference in its entirety. The results of the binding affinity for brain serotonin 5-HT₂ receptor and for dopamine D₁ and D₂ receptors are ascertained. It is postulated that the combination of serotonin and dopamine receptor antagonism, with higher relative 5-HT₂ to D₂ receptor affinity that indicates the compound as a potent atypical antipsychotic. J. Goldstein, “Quetiapine Fumarate (Seroquel): a new atypical antipsychotic,” 35(2) Drugs of Today 1993-210 (1999), which is incorporated herein by reference in its entirety.

Additionally, in vivo antipsychotic activity of the compounds and compositions of the invention can be tested in mice according to the standard apomorphine climbing mouse assay (see e.g. Strupczewski et. al., J. Med. Chem., 1995, 38, 1119).

### Example 4

### Alpha Receptor Binding Assays

The compounds and compositions of the invention can be compared with quetiapine based on alpha receptor binding assays using the following receptors.

| | Quetiapine | (the compounds of invention) |
|---|---|---|
| Receptor | Affinity (nM) | Affinity (nM) |
| α1A | 22 | — |
| α1B | 39 | — |
| α1D | — | — |
| α2C | 28.9 | — |

The affinity values can be derived from the below methods and criteria.

TABLE 1

| PRIMARY | | | |
|---|---|---|---|
| BIOCHEMICAL | | | |
| ASSAY | SPECIES | CONC. | |
| Adrenergic α_{1A} | rat | 0.3 | μM |
| Adrenergic α_{1B} | rat | 0.1 | μM |
| Adrenergic α_{1D} | hum | 0.3 | μM |
| Adrenergic α_{2A} | hum | 3 | μM |
| Adrenergic α_{2B} | hum | 1 | μM |
| Adrenergic α_{2C} | hum | 10 | μM |
| Adrenergic α₁* | rat | 0.1 | μM |
| Adrenergic α₂* | rat | 10 | μM |

Receptor binding methods, α-adrenergic subtype specific, are provided below.

203100 Adrenergic α_{1A}

| | |
|---|---|
| Source: | Wistar Rat submaxillary gland |
| Ligand: | 0.25 nM [³H] Prazosin |
| Vehicle: | 1% DMSO |
| Incubation Time/Temp: | 60 minutes @ 25° C. |
| Incubation Buffer: | 20 mM Tris-HCI, 0.5 mM EDTA, pH 7.4 |
| Non-Specific Ligand: | 10 μM Phentolamine |
| K_{D}: | 0.17 nM * |
| B_{MAX}: | 0.18 pmole/mg Protein * |
| Specific Binding: | 90% * |
| Quantitation Method: | Radioligand Binding |
| Significance Criteria: | ≧50% of max stimulation or inhibition |

203200 Adrenergic α_{1B}

| | |
|---|---|
| Source: | Wistar Rat liver |
| Ligand: | 0.25 nM [³H] Prazosin |
| Vehicle: | 1% DMSO |
| Incubation Time/Temp: | 60 minutes @ 25° C. |
| Incubation Buffer: | 20 mM Tris-HCI, 0.5 mM EDTA, pH 7.4 |
| Non-Specific Ligand: | 10 μM Phentolamine |
| K_{D}: | 0.31 nM * |
| B_{MAX}: | 0.18 pmole/mg Protein * |
| Specific Binding: | 90% * |
| Quantitation Method: | Radioligand Binding |
| Significance Criteria: | ≧50% of max stimulation or inhibition |

203400 Adrenergic α_{1D}

| | |
|---|---|
| Source: | Human recombinant HEK-293 cells |
| Ligand: | 0.6 nM [³H] Prazosin |
| Vehicle: | 1% DMSO |
| Incubation Time/Temp: | 60 minutes @ 25° C. |
| Incubation Buffer: | 50 mM Tris-HCI |
| Non-Specific Ligand: | 10 μM Phentolamine |
| K_{D}: | 0.58 nM * |
| B_{MAX}: | 0.17 pmole/mg Protein * |
| Specific Binding: | 80% * |
| Quantitation Method: | Radioligand Binding |
| Significance Criteria: | ≧50% of max stimulation or inhibition |

Receptor binding methods, α-adrenergic nonselective, are provided below.

203500 Adrenergic α₁* Non-Selective

| | |
|---|---|
| Source: | Wistar Rat brain |
| Ligand: | 0.25 nM [³H] |
| | Prazosin |
| Vehicle: | 1% DMSO |
| Incubation Time/Temp: | 30 minutes @ 25° C. |
| Incubation Buffer: | 50 mM Tris-HCI, 0.1% ascorbic acid |
| | 10 μM pargyline |
| Non-Specific Ligand: | 0.1 μM Prazosin |
| K_{D}: | 0.29 nM * |
| B_{MAX}: | 0.095 pmole/mg Protein * |
| Specific Binding: | 90% * |
| Quantitation Method: | Radioligand Binding |
| Significance Criteria: | ≧50% of max stimulation or inhibition |

203900 Adrenergic α₂* Non-Selective

| | |
|---|---|
| Source: | Wistar Rat cerebral cortex |
| Ligand: | 0.7 nM [³H] |
| | Prazosin |
| Vehicle: | 1% DMSO |
| Incubation Time/Temp: | 30 minutes @ 25° C. |
| Incubation Buffer: | 20 mM Hepes, 2.5 mM Tris-HCI, |
| | pH 7.4 @ 25° C. |
| Non-Specific Ligand: | 1 μM Yohimbine |
| K_{D}: | 7.8 nM * |
| B_{MAX}: | 0.36 pmole/mg Protein * |
| Specific Binding: | 80% * |
| Quantitation Method: | Radioligand Binding |
| Significance Criteria: | ≧50% of max stimulation or inhibition |

### Example 5

### In vivo Anxiolytic Assays

Anxiolytic activity of the compounds and compositions of the invention can be tested in rats according to the Geller-Seifter conflict test.

Subjects: 30 Male Long Evans rats are used. Subjects weigh 350-450 g at the time of testing, and are food restricted to 85% of free feeding weight by post session feeding with approximately 15 g of standard rat chow per day. All animals have free access to water except during experimental testing. Subjects are individually housed throughout the course of the experiment under a 12 hr light/dark cycle.

Apparatus: Standard 2-lever operant chambers are used (Med Associates). The chambers are fitted with two retractable response levers and a stimulus lamp over each of the 2 levers. A pellet food dispenser delivers 45 mg food pellets, (Bio Serv) to a cup located inside of the chamber below and between the 2 response levers. A lamp at the top and back of the chamber serves as houselights. The grid floors of the operant chambers are interfaced to shock generators and scramblers (Med Associates). All events in the chambers are controlled and monitored by a microprocessor.

Procedure: There are two components in the procedure: 1) unsuppressed responding components (unpunished) with 2 minutes in duration and 2) suppressed responding components (punished) with 3 minutes in duration. In unpunished components, the houselights and both stimulus lamps over the response levers are turned on, the lever on the left-hand side of the chamber extends, and a food pellet is delivered following an average of 17 responses on the lever in the chamber (range 3 to 40 responses)—a variable ratio 17 schedule (VR17). The punished components follow unpunished components, and during these, the right-hand lever is extended into the chamber, and the stimulus lamps and houselights are turned on and off at 1 s intervals, in succession, which serve as a cue for this component. In the punished component, food is also available under a VR17 schedule, but in addition, electrical current (0.5 s duration) is delivered to the grid floor of the chamber under an independent VR17 schedule. The level of the current is adjusted for each individual subject until responding is reduced in the suppressed component to a level that is about 5-10% that of the unpunished component, and ranges from 0.2 mA to 0.75 mA. Unpunished and punished components are separated by 10 s time-out periods in which both response levers are retracted and all stimulus lamps turn off. 2-Min unpunished and 3-min unpunished components alternate until 5 of each are completed. Daily sessions always begin with unpunished responding component.

Rats whose responding is most stable are chosen from a larger pool of trained rats. Several doses are tested on a given day in different subjects. Each dose, then, is tested in a different sub-set of rats. The dependent variables recorded are the rate of responding in unpunished and punished components (total responses/total time under the component), the number of shocks delivered. A selective anxiolytic effect is defined as an increase in responding in the unpunished components with relatively less or no effect on responding in unpunished components. t-Tests are used to compare mean of the control's rate of responding on vehicle day of the rats used for a specific dose to the same rats means following delivery of each dose of compound (for only the rats used within each dose). Brains, CSF and plasma are collected in a satellite group of rats that match the Geller-Seifter rats to evaluate exposure levels.

Test compounds: Once animals are trained to a stable baseline for 3 consecutive days, the testing begin. Test compounds are administered on Tuesdays and Fridays s.c. in a volume of 1 mL/kg. Doses of 0.3, 1, 2, 5, and 10 mg/kg are dissolved in saline and the highest stock solution is prepared, and appropriate concentration prepared by serial dilutions into saline. Diazepam (for comparison purposes) can be supplied in an Abbott's cocktail (10% ethanol, 40% propylene glycol, 50% water) solution in a concentration of 5 mg/mL, and is prepared by serial dilution (0.3, 1, and 3 mg/kg) into 50% concentration of Abbott's cocktail. The test compound has a 15 minute pre-treatment time whereas diazepam is dosed 30 min prior to testing. On average 6-10 rats are for each dose of drug and 3-5 for the diazepam.

Exposure sampling: In weight and feeding status-matched subjects, terminal plasma, whole brain and CSF samples are collected. Four rats are used for each of the 4 doses of the test compound, with samples obtained 15 min after dosing.

Statistics: Absolute rate of responding in punished and unpunished components is the endpoint measured for individual subjects, and the means reported. The % control rate of responding is calculated as the (rate of responding following drug administration/rate following vehicle administration)×100. This calculation is performed for individual subjects, and the means reported. The Student t-Test is used to compare mean control rates for a given set of rats to their corresponding rate of responding after the test compound administration.

### Example 6

### In vivo Antipsychotic Activity of 11-piperazin-1-yldibenzo[b,f][1,4]thiazepine

Antipsychotic activity of the compounds and compositions of the invention can be tested in rats according to the D-amphetamine locomotor activity test.

Locomotor activity (LMA) can be assessed in male Long Evans rats using a paradigm that included a habituation phase followed by administration of D-amphetamine at various doses. Animals are allowed to acclimatize to the testing room for 1 hour before being weighed and placed into activity chambers. Forty-five min after LMA measurements begin, animals are briefly removed, dosed with drug (1, 2, 5, or 10 mg/kg) or vehicle at 1 mL/kg and returned to the chambers. After a further 15 min animals are again removed and dosed with vehicle or D-amphetamine at 1 mg/kg via s.c. route. After returning the animals to the activity chambers, LMA is assessed for a further 60 min. Statistical analysis is made of total distance traveled after D-amphetamine administration using ANOVA and Tukey's post hoc analysis where appropriate.

### Example 7

### In vivo Antidepressant Activity Assays

Antidepressant activity of the compounds and compositions of the invention can be assessed in mice according to the tail suspension test.

In a first experiment, mice (n=10/group) are treated with vehicle, 2.0, 5.0, or 10.0 mg/kg of the test compound s.c. 15 minutes prior to the test session. Mice are suspended from their tails for 7 minutes. During the last 5 min of the test, the duration of immobility is recorded. In a second experiment, mice (n=10/group) are treated with vehicle, 30 mg/kg of the test compound, or 30 mg/kg of fluoxetine p.o. 60 minutes prior to the test session. Mice are suspended from their tails for 7 minutes. During the last 5 minutes of the test, the duration of immobility is recorded.

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety. U.S. Application Ser. No. 60/870,982 is incorporated herein by reference in its entirety.

1. A compound of Formula I: 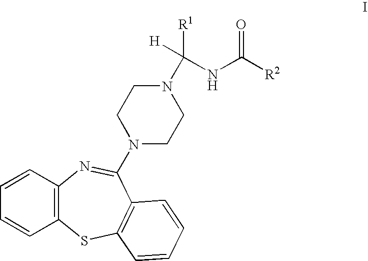
or a pharmaceutically acceptable salt thereof, wherein:
R¹ is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R³;
R² is H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalky is optionally substituted by 1, 2, 3, 4, or 5 R⁴;
each R³ is, independently, halo, C₁₋₆ alkyl, C₁₋₆ haloalkyl, aryl, cycloalkyl, heteroaryl, heterocycloalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c′}S(═O)₂R^{b}, S(═O)R^{b}, S(═O)NR^{c}R^{d}, S(═O)₂R^{b}, or S(═O)₂NR^{c}R^{d};
each R⁴ is, independently, halo, C₁₋₆ alkyl, C₁₋₆ haloalkyl, aryl, cycloalkyl, heteroaryl, heterocycloalkyl, CN, NO₂, OR^{a′}, SR^{a′}, C(═O)R^{b′}, C(═O)NR^{c′}R^{d′}, C(═O)OR^{a′}, OC(═O)R^{b′}, OC(═O)NR^{c′}R^{d′}, NR^{c′}R^{d′}, NR^{c′}C(═O)R^{b′}, NR^{c′}C(═O)OR^{a′}, NR^{c′}S(═O)₂R^{b′}, S(═O)R^{b′}, S(═O)NR^{c′}R^{d′}, S(═O)₂R^{b′}, or S(═O)₂NR^{c′}R^{d′};
each R^{a} and R^{a′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
each R^{b} and R^{b′} is, independently, selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
each R^{c} and R^{d} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{c} and R^{d} together with the N atom to which they are attached form a 4-, 5-, 6-, or 7-membered heterocycloalkyl group; and
each R^{c′} and R^{d′} is, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, C₁₋₆ alkoxy, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{c′} and R^{d′} together with the N atom to which they are attached form a 4-, 5-, 6-, or 7-membered heterocycloalkyl group.

2-36. (canceled)

37. A compound according to claim 1 wherein R¹ is H, C₁₋₄ alkyl, or C₁₋₄ haloalkyl; and R² is C₁₋₄ alkyl.

38. A compound according to claim 1 wherein R¹ is H, C₁₋₄ alkyl, or C₁₋₄ perfluoroalkyl; R² is NHC(═O)—R³; and R³ is C₁₋₄ alkyl.

39. A pharmaceutical composition comprising a compound according to claim 1, and further comprising a pharmaceutically acceptable carrier.

40. The composition of claim 39 further comprising at least one antidepressant, atypical antipsychotic, antipsychotic, anxiolytic, anticonvulsant, Alzheimer's therapy, Parkinson's therapy, migraine therapy, stroke therapy, urinary incontinence therapy, neuropathic pain therapy, nociceptive pain therapy, insomnia therapy, mood stabilizer, 5HT_{1B} ligand, mGluR2 agonist, alpha 7 nicotinic agonist, chemokine receptor CCR1 inhibitor, or delta opioid agonist.

41. The composition of claim 39 further comprising at least one benzodiazepine, 5-HT_{1A} ligand, 5-HT_{1B} ligand, 5-HT_{1D} ligand, mGluR2A agonist, mGluR5 antagonist, antipsychotic, NK1 receptor antagonist, antidepressant, or serotonin reuptake inhibitor.

42. A method of treating at least one symptom or condition associated with schizophrenia and other psychotic disorder, dementia and other cognitive disorder, anxiety disorder, mood disorder, sleep disorder, disorder usually first diagnosed in infancy, childhood, or adolescence, or neurodegenerative disorder, comprising administering to a mammal a therapeutically effective amount of a compound according to claim 1.

43. The method of claim 42 wherein said symptom or condition comprises anxiety, agitation, hostility, panic, eating disorder, affective symptom, mood symptom, or negative or positive psychotic symptom.

44. A method of treating schizophrenia comprising administering a therapeutically effective amount of a compound according to claim 1 to a subject in need thereof.
